(19) United States
(12) Patent Application Publication
(10) Pub. No.: US 2003/0020874 A1
(43) Pub. Date: Jan. 30, 2003

(54) Adaptive ablation centering for pupil dilation effects

(76) Inventors: Michael J. Smith, Orlando, FL (US); Lance R. Marrou, Orlando, FL (US)

Correspondence Address: MANELLI DENISON & SELTER PLLC, 7th Floor, 2000 M Street, N.W., Washington, DC, 20036-3307, US

(21) Appl. No.: 10/207,130

(22) Filed: Jul. 30, 2002

Related U.S. Application Data
(60) Provisional application No. 60/308,130, filed on Jul. 30, 2001.

(51) Int. Cl. A61B 3/10; A61B 3/14

(52) U.S. Cl. 351/204; 351/209

(57) ABSTRACT

A method and apparatus to detect a center of a visual axis (or any spot on the pupil) that can adapt to a varying pupil size. Due to the dynamic nature of the human pupil that changes location registration with the visual axis with changes in the pupil size, the following method is provided as a means to remove positional errors induced by this cause. The visual axis correction is provided by means of a functional estimation using linear, polynomial, parametric or piece-wise functional estimation. Methods for determining this estimator and the typical uses of the estimator are provided in the invention specification.

This application is based on and claims priority from U.S. Provisional Application No. 60/308,130 filed on Jul. 30, 2001, the entirety of which is expressly incorporated herein by reference.

### BACKGROUND OF THE INVENTION

1. Field of the Invention

The invention relates to a method for correcting the visual axis center location with respect to the pupil center and a method for generating a visual axis center characterization function. This invention applies to refractive surgery using eye tracking techniques and other systems which use human pupil tracking.

2. Background

Based on experimental data from various researchers in the ophthalmic field, including such individuals as Walsh (1988) and Wilson (1992) as reported in the book Optics of the Human Eye by Atchison and Smith (2000) pg. 23, the pupil center can move up to 0.4 mm based on dilation (See FIGS. 4A to 4C). This means that a refractive treatment targeted on the cornea based upon the pupil center during one light condition (i.e. scotopic in low lighting conditions) will be different than in another light condition (i.e. photopic in high lighting conditions). Additional data may suggest that the maximum center movements can be greater than 0.4 mm for certain individuals. These differences in the pupil center with respect to the cornea can present problems for human gaze tracking or refractive surgery procedures that must be registered to the gaze center.

The problems related to this human pupil characteristic and a method to account for it will be presented in this patent specification. This method covers a means for determining the pupil center under multiple pupil sizes and a means to use this information for gaze tracking or proper detection of the pupil center.

### SUMMARY OF THE INVENTION

An object of the invention is to provide a method to reduce the error in locating the visual axis center with respect to the pupil center. This objective is achieved by another object of this invention, which is providing a method to construct a functional relation between the visual axis change with pupil size and providing a method to use this function to correct visual axis changes for newly sampled pupil locations and sizes. The determination of the functional relationship between the visual axis changes with pupil size is described in FIG. 1. FIGS. 3A and 3B represent a hypothetical set of functional descriptions of the visual axis center offset from the pupil center used later during the correction of newly sampled pupil center locations. Using these methods, the resulting tracking error can be reduced to allow for more precise measurements of the current visual axis center.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, in which:

FIG. 1 is an exemplary flow chart showing a technique for generating the visual axis center characterization function. Possible variations in number of sampled light levels and fitting functions can be extrapolated from this flow chart.

FIG. 2 is a flow chart showing the technique for using the generated visual axis center characterization function to correct the visual axis center for pupil dilation effects.

FIGS. 3A and 3B are an exemplary set of graphs showing hypothetical X and Y plots of the pupil size parameter to the X and Y offset (Cartesian). Polar, parametric, or similar functions may be used instead of strictly Cartesian offsets.

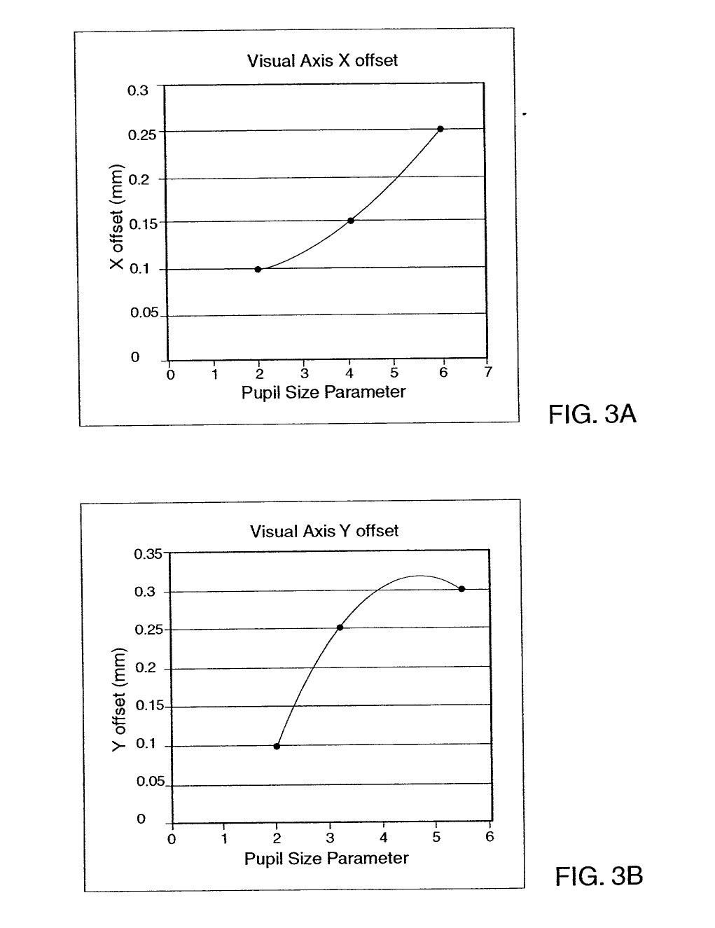

FIGS. 4A to 4C are exemplary plan views depicting the typical difference from low to moderate to high lighting conditions for the pupil centers and visual axis centers.

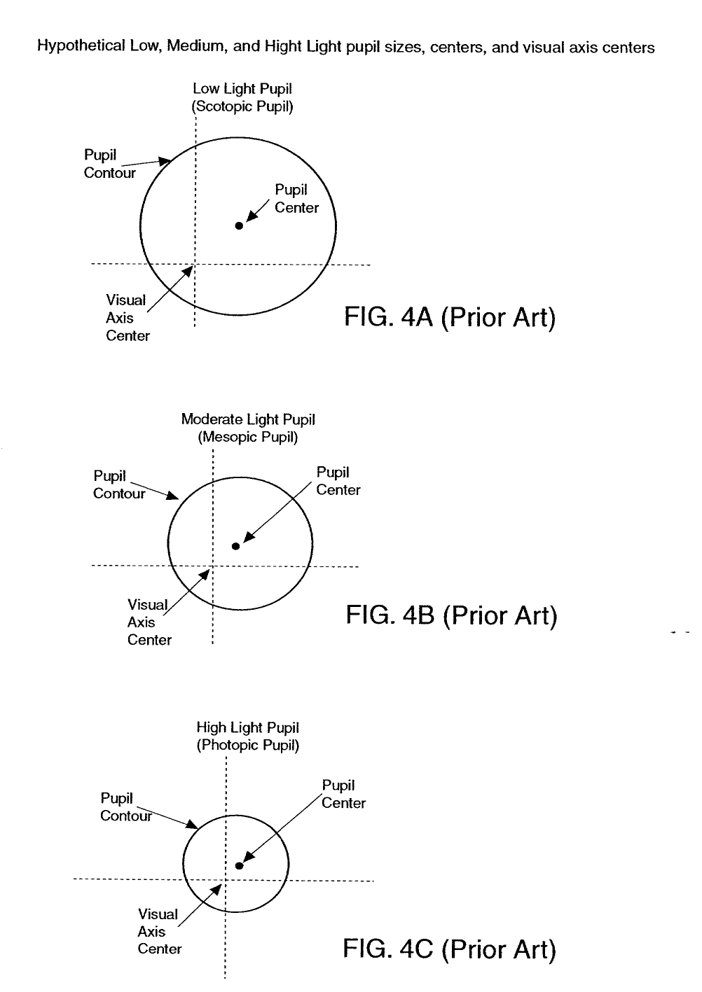

### DETAILED DESCRIPTION OF ILLUSTRATIVE EMBODIMENTS

Visual Axis Center Characterization Function

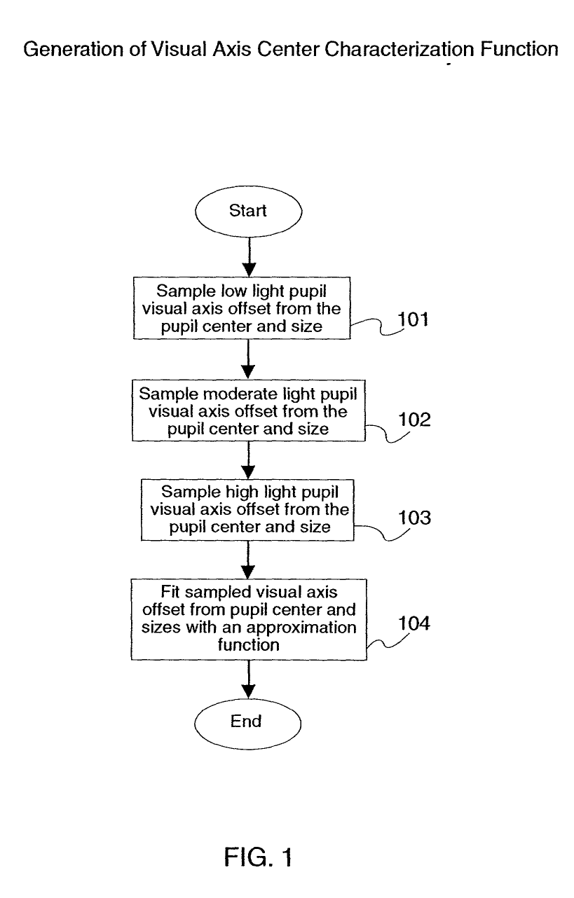

For representation of any point on the cornea it is sufficient to determine any single point that is invariant, and reference this point to determine offsets from this point. For simplicity, the visual axis center location will be used as a reference to determine the pupil dilation error function, but other reference points would also be appropriate. A straightforward method to apply this technique would be to determine the pupil center offset from the visual axis center for the pupil under three basic lighting conditions: high light, moderate light, and low light. A function can then be fit to the pupil center offset from the visual axis center that provides a visual axis offset for any given pupil size. Other similar methods may also be used which sample more points, such as an automated exam which starts at low-light conditions and continuously samples the pupil size as the light level is increased, constricting the pupil. Linear, polynomial, parametric or piece-wise functions can then be fit to the visual axis offset from pupil center values. With this, given any pupil size parameter an offset from the pupil center (or centroid) can be determined to locate the visual axis center location. This method is outlined in FIG. 1. This function under this example is patient-specific, but a sample from a similar population base may be utilized to determine the visual axis center characterization function for the average population.

Correction of the Visual Axis Center During Pupil Tracking

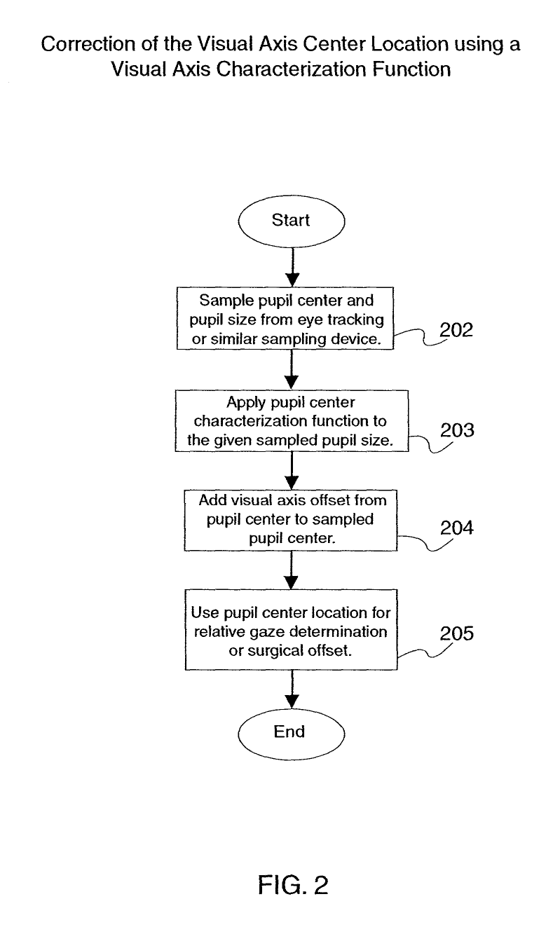

For procedures such as refractive surgery, accurate gaze tracking is required to produce the best results. A common means of tracking the human gaze is to use infrared light and track the pupil aperture. However, other similar eye tracking methods may benefit from the present invention. To determine the estimated visual axis center (gaze center), the visual axis center characterization function would be applied to the current pupil size parameter and added to the pupil center location. This method is described in FIG. 2. By using this method during refractive surgery, the pupil dilation effects from the physician's chosen light level will not affect the surgical registration. This registration is especially important for custom refractive surgeries where the centering requirements are higher than for basic treatments. This would also benefit other gaze-tracking systems that must operate under differing light levels.

The device is not limited to refractive surgery and may be used in other applications such as for more accurate gaze tracking for other medical devices, games, heads-up displays, or gaze selection devices.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the claims in the following claims section.

### DEFINITIONS

Gaze Center—Generally considered the same as the visual axis center.

Gaze Tracking System—Any system which tracks the human eye and attempts to determine the direction the subject is looking. This can be useful for determining what objects people are looking at in an environment, or for devices that use this as a form of input.

Function—Relationship meant to map independent variables to dependent variables. Functions can be expressed as point relations, linear relations, polynomial relations, parametric relations, or piece-wise combinations of any of the previously mentioned relations.

Mesopic Pupil Size—The pupil size under moderate lighting that is between normal reading light and night driving conditions.

Photopic Pupil Size—The pupil size under normal sunlight, high lighting, or reading conditions.

Pupil Size Parameter—A metric of the pupil size that may be characterized by the radius, x diameter, y diameter, function of x and y diameter or radii, area, or other similarly computed composite metric.

Pupil Center—A measurement denoting the center of the apparent pupil. This may be the centroid of the pupil image, a geometric center (i.e. circle or ellipse estimated center), or a parametric center (i.e. center of a parametric equation for the pupil contour).

Refractive Surgery—Surgery performed to bring about a refractive change in the human vision system to account for vision problems that require glasses to achieve normal vision or correct corneal blindness not correctable by glasses.

Scotopic Pupil Size—The pupil size under low light or night driving conditions.

Visual Axis Center—The location through which a person apparently looks out of their eye. This location is normally located close to the center of the pupil. This is generally used for reference as the gaze center.

Visual Axis Center Characterization Function—A function that produces the offset from the pupil center to the visual axis for a given pupil size parameter. The offset function may be a linear, polynomial, parametric, or piece-wise combination of any of the aforementioned function types. Also, the offset may be Cartesian, polar, parametric or similar functions that provide an offset from the sampled pupil center location in similar coordinate systems.

What is claimed is:

1. A method of correcting for a visual axis center location, said method comprising:
sampling a first pupil center and a first pupil size at a first light level;
sampling a second pupil center and a second pupil size at a second light level;
applying said first pupil center and first pupil size at said first light level, and applying said second pupil center and said second pupil size at said second light level, to a pupil center characterization function; and
adding a visual axis offset to a sampled pupil center.

2. A method of correcting for a visual axis center location, said method further comprising:
creating an offset function to estimate a location of a pupil center for any given pupil size.

3. A method of generating a visual axis center characterization function, said method comprising:
sampling a plurality of pupil centers versus pupil sizes; and
creating an offset function to estimate a location of a pupil center for any given pupil size.

4. A method of generating a visual axis center characterization function according to claim 3, wherein parameters of said pupil sizes can be at least one of the following:
pupil radius;
pupil diameter;
pupil horizontal diameter;
pupil vertical diameter;
a function of pupil horizontal and vertical radius;
a function of pupil horizontal and vertical diameters;
pupil area;
pupil contour arc length; and
current pupil light level.

5. A method of generating a visual axis center characterization function according to claim 3, wherein said offset function is one of:
a linear function;
a polynomial;
a parametric function; and
a piece-wise function.

6. A method of generating a visual axis center characterization function according to claim 3, wherein:
said offset function returns at least one of Cartesian, polar, and parametric offsets for addition to a sampled pupil center location.

7. Apparatus for correcting for a visual axis center location, said method comprising:
means for sampling a first pupil center and a first pupil size at a first light level;
means for sampling a second pupil center and a second pupil size at a second light level;
means for applying said first pupil center and first pupil size at said first light level, and applying said second pupil center and said second pupil size at said second light level, to a pupil center characterization function; and
means for adding a visual axis offset to a sampled pupil center.

8. Apparatus for correcting for a visual axis center location, said method further comprising:
means for creating an offset function to estimate a location of a pupil center for any given pupil size.

9. Apparatus for generating a visual axis center characterization function, said method comprising:
means for sampling a plurality of pupil centers versus pupil sizes; and
means for creating an offset function to estimate a location of a pupil center for any given pupil size.

10. Apparatus for generating a visual axis center characterization function according to claim 9, wherein parameters of said pupil sizes can be at least one of the following:
pupil radius;
pupil diameter;
pupil horizontal diameter;
pupil vertical diameter;
a function of pupil horizontal and vertical radius;
a function of pupil horizontal and vertical diameters;
pupil area;
pupil contour arc length; and
current pupil light level.

11. Apparatus for generating a visual axis center characterization function according to claim 9, wherein said offset function is one of:
a linear function;
a polynomial;
a parametric function; and
a piece-wise function.

12. Apparatus for generating a visual axis center characterization function according to claim 9, wherein:
said means for creating an offset function returns at least one of Cartesian, polar, and parametric offsets for addition to a sampled pupil center location.
